(19) United States
(12) Patent Application Publication
(10) Pub. No.: US 2024/0290477 A1
(43) Pub. Date: Aug. 29, 2024

(54) AUTOMATED DISPENSING CABINET TRIP MANAGEMENT SYSTEM

(71) Applicant: CAREFUSION 303, INC., San Diego, CA (US)

(72) Inventors: Dennis Tribble, Ormond Beach, FL (US); Cynthia Yamaga, Oceanside, CA (US); Roxana Brown, Solana Beach, CA (US); Saeedeh Abbasi, San Diego, CA (US)

(21) Appl. No.: 18/571,117

(22) PCT Filed: May 24, 2022
(86) PCT No.: PCT/US22/30737

§ 371 Date: Dec. 15, 2023

Related U.S. Application Data
(60) Provisional application No. 63/215,342, filed on Jun. 25, 2021.

(51) Int. Cl. G16H 40/20; G05D 1/667; G05D 105/20; G05D 107/60; G16H 20/13; G16H 50/20

(52) U.S. Cl. CPC G16H 40/20; G05D 1/667; G16H 20/13; G16H 50/20; G05D 2105/20; G05D 2107/65

(57) ABSTRACT

A method for managing trips from a pharmacy may include receiving a plurality of transaction records that, as received, represent an independent transaction of a plurality of transactions at an ADC. The plurality of transaction records may include: time information between respective transactions and an elapsed time for a portion of the plurality of transactions. The method may include identifying, based at least in part on: (i) a comparison of the time information between respective transactions and a threshold inter-transaction trip time; and (ii) a comparison of the elapsed time for the portion and a threshold elapsed trip time, a sequence of the portion as a unique trip from a pharmacy. The method may include annotating, with an identifier for the unique trip, a portion of the plurality of transaction records representing the portion of the plurality of transactions. Related methods and articles of manufacture are also disclosed.

### CROSS-REFERENCE TO RELATED APPLICATIONS

The present application claims priority to U.S. Provisional Application No. 63/215,342, filed Jun. 25, 2021, and entitled “Trip Management System,” the entirety of which is incorporated by reference herein.

### TECHNICAL FIELD

The subject matter described herein relates to the management of medication delivery trips from pharmacies to dispensing cabinets at a medical facility.

### BACKGROUND

Institutional pharmacies use automated dispensing cabinets (“ADCs”) to provide convenient access to frequently-used medication for caregivers in patient care areas. Unique medications are stored in unique storage locations (hereinafter referred to as “pockets”) of the ADCs, whose inventory quantities are governed by minimum and maximum values that, among other things, stimulate requests for refill.

Generally, maintaining the ADCs may include refilling current medication supplies, adding new medication supplies, removing medication supplies that are no longer needed, performing inventory count verifications, checking for expired medications, removing expired medications, relocating soon-to-expire medications to areas where they are more likely to be consumed before they expire, and so on. These functions are generally performed by pharmacy personnel (hereinafter referred to as “couriers”).

As part of their daily routine, medical professionals, such as the couriers, make trips to patient care areas to perform a number of these maintenance transactions at one or more ADCs. For example, a transaction may include adding a new medication to an ADC (e.g., loading), removing medication that is no longer used from the ADC (e.g., unloading), removing medication from one ADC and transferring the medication to another ADC (e.g., destocking), taking inventory of pockets in the ADC (e.g., inventory), refilling a pocket of the ADC (e.g., refilling), emptying a return bin of the ADC, removing expired medications from the ADC, and/or the like. Some medical facilities generate tens or hundreds of thousands of transaction records per month when transactions are performed at the ADCs.

A need exists to efficiently identify, in some instances in real-time, which transactions are part of a single trip. The trips taken by the couriers to perform such transactions may be planned or scheduled, or may be unplanned or ad hoc. In some instances, all or a portion of scheduled trips may be inefficient, redundant, and/or unnecessary. For example, couriers may take poorly planned routes to perform the transactions at each ADC, medications stored at the ADC may not yet need to be refilled at the time of the scheduled trip, a trip to remove expired medications from a pocket when all the expired medications had been consumed and none need to be removed, or the like. Additionally, ad hoc trips, often stemming from inefficient scheduled trips, may be time consuming, may interfere with other tasks performed by the couriers, and may interfere with the administration of medications to patients. A further need exists for identifying which trips are scheduled trips and which trips are ad hoc. Nevertheless, systems generally do not document which trips are ad hoc. Therefore, pharmacy managers can therefore not assess, much less manage those trips.

### SUMMARY

A method may include receiving a plurality of transaction records. Each transaction record of the plurality of transaction records, as received, represents an independent transaction of a plurality of transactions at an automated dispensing cabinet (ADC) at a medical facility. The plurality of transaction records may include: time information between respective transactions of the plurality of transaction and an elapsed time for a portion of the plurality of transactions. The method may also include identifying, based at least in part on: (i) a comparison of the time information between respective transactions of the plurality of transactions and a threshold inter-transaction trip time; and (ii) a comparison of the elapsed time for the portion of the plurality of transactions and a threshold elapsed trip time, a sequence of the portion of the plurality of transactions as a unique trip from a pharmacy. The method may also include annotating, with an identifier for the unique trip, a portion of the plurality of transaction records representing the portion of the plurality of transactions.

In some aspects, the method may include predicting, based on the identification of the unique trip and a model that accepts a set of input values characterizing a trip and provides a set of output values identifying a trip type of the trip, the trip type of the unique trip. The trip type of the unique trip is at least one of a scheduled trip or an unscheduled trip.

In some aspects, the method may include annotating, with an identifier of the trip type, at least one of the portion of the plurality of transaction records and a record for the unique trip.

In some aspects, the method may include scheduling, based in part on the trip type, at least a portion of a future trip from the pharmacy to the ADC.

In some aspects, the method may include filtering, based on the identifier of the trip type, a plurality of trip records. Each of the plurality of trip records represents an independent trip.

In some aspects, the filtering includes: removing, from the plurality of trip records, at least one trip record corresponding to the identifier of the trip type.

In some aspects, the method may include determining a total number of trips having the trip type over a predetermined period of time. The method may include detecting that the total number of trips over the predetermined period of time is greater than or equal to a threshold number of trips. The method may include generating an alert indicating that the total number of trips over the predetermined period of time is greater than the threshold number of trips.

In some aspects, the method may include adjusting, based on the detection, a schedule of a future trip.

In some aspects, the method may include causing presentation of a first plot of trips representing a plurality of trips at the medical facility, the plurality of trips comprising the unique trip. The method may include receiving a message requesting a second plot of trips showing at least two trip types for the medical facility, the at least two trip types comprising an unscheduled trip and a scheduled trip. The method may include predicting, based on the identification of the unique trip and a model that accepts a set of input values characterizing a trip and provides a set of output values identifying a trip type for of the trip, the trip type of each trip of the plurality of trips. The method may include causing presentation of the second plot of trips. The second plot of trips may include: a first visualization for at least one trip of the plurality of trips identified as the unscheduled trip; and a second visualization for at least one trip of the plurality of trips identified as the scheduled trip.

In some aspects, the identifying may also include: dynamically generating, based on one or more of historical trip data, a size of the medical facility, a number of patient rooms at the medical facility, a number of ADCs at the medical facility, a distance from a pharmacy to a farthest possible location of a transaction of the plurality of transactions, a square footage of the medical facility, a number of patients being treated at the medical facility, a number of elevators at the medical facility, and a number of floors at the medical facility, the inter-transaction threshold.

In some aspects, the identifying further includes: dynamically generating, based on one or more of historical trip data, a size of the medical facility, a number of patient rooms at the medical facility, a number of ADCs at the medical facility, a distance from a pharmacy to a farthest possible location of a transaction of the plurality of transactions, a square footage of the medical facility, a number of patients being treated at the medical facility, a number of elevators at the medical facility, and a number of floors at the medical facility, the threshold elapsed trip time.

In some aspects, the method may include generating, based on the portion of the plurality of transaction records, at least one trip metric indicating an effectiveness of the unique trip. The method may include displaying, based on the at least one trip metric, a visualization indicating the effectiveness of a medical workflow comprising a plurality of trips. The plurality of trips may include the unique trip.

In some aspects, the method may include triggering, based on the at least one trip metric and/or the visualization, an investigative workflow.

In some aspects, the investigative workflow is triggered in response to a determination that the at least one trip metric is greater than or equal to a threshold.

In some aspects, the model includes a machine-learning model trained to identify the trip type.

In some aspects, the determination of the trip type of the unique trip is configured to enable an improved medical workflow.

In some aspects, the at least one trip metric comprises one or more of: a duration of at least one trip of the plurality of trips, a quantity of ADCs visited during the at least one trip, a transaction type of the transaction performed at the ADC during the at least one trip, and a quantity of different transaction types of the transaction performed at the ADC during the at least one trip, a total quantity of trips in the medical workflow, a total quantity of refill transactions performed during the medical workflow, a total quantity of critical low refill transactions performed during the medical workflow, a total quantity of refills less than maximum transactions performed during the medical workflow, a total quantity of inventory transactions performed during the medical workflow, a total quantity of load and/or unload transactions performed during the medical workflow, a total quantity of expiration no-remove transactions performed during the medical workflow, a total quantity of expiration remove transactions performed during the medical workflow, a total duration of the medical workflow, a total quantity of transactions performed during the medical workflow, and a total quantity of stock out refills transactions performed during the medical workflow.

In some aspects, the method may include suggesting, based on the at least one trip metric and/or the visualization, an updated medical workflow configured to reduce a quantity of unscheduled trips of the plurality of trips of the medical workflow.

In some aspects, the method may include suggesting, based on the at least one trip metric and/or the visualization, an updated medical workflow. The updated medical workflow may include one or more of: a sequence of the plurality of trips of the medical workflow, a time at which the plurality of trips should be performed, one or more transactions to perform during each of the plurality of trips, and a route through the medical facility.

In some aspects, the method may include suggesting, based on the at least one trip metric and/or the visualization, an updated medical workflow. The updated medical workflow may include a route for a robot to deliver a medication to the ADC.

In some aspects, the method may include identifying, based on the plurality of transaction records, a possible diversion of medication. The method may include triggering, based on the identification of the possible diversion, an investigative workflow.

In some aspects, the investigative workflow includes sending, to a client device, an alert indicating the possible diversion.

In some aspects, the investigative workflow includes activating, at the ADC, one or more surveillance devices in response to an interaction between a medical professional suspected of the possible diversion and the ADC.

In some aspects, the identification of the possible diversion is further based on a determination of an anomalous route during the medical workflow.

In some aspects, the plurality of transaction records are generated from one or more data systems comprising the ADC, an access control system, and/or an infusion system.

In some aspects, the plurality of transaction records are generated from one or more of the ADC and an identification tag of a medical professional performing the independent transaction.

In some aspects, the predicting includes: determining an inflection value from the set of input values.

In some aspects, the predicting further includes: determining that the unique trip is the scheduled trip when the inflection value is greater than or equal to a threshold value; and determining that the unique trip is the ad hoc trip when the inflection value is less than the threshold value.

In some aspects, the plurality of transaction records includes one or more transaction values corresponding to a timestamp, a patient identifier, a device identifier, a clinician identifier, a medication identifier, a prescription order identifier, an inventory information, a patient status, a shift identifier, a location tracking identifier, an infusion information, a compounding information, an administration information, a working off clock indicator, and/or an electronic health record identifier.

In some aspects, the plurality of transaction records includes one or more transaction values corresponding to a start time of the unique trip, a stop time of the unique trip, a quantity of transactions performed at the ADC, a quantity of ADCs visited during the unique trip, an identity of each ADC visited during the unique trip, an identity of each transaction type performed at the ADC, one or more refill parameters, and one or more medication removal parameters.

In some aspects, the one or more refill parameters includes one or more of a number of refills, a number of refills to pockets of the ADC that are empty, a number of refills to pockets of the ADC that are below a minimum PAR value, a number of refills that do not completely refill a corresponding pocket of the ADC to a maximum PAR value, a beginning count of the medication in the pockets of the ADC, an end count of the medication in the pockets of the ADC, and a quantity of the medication in the pockets of the ADC.

In some aspects, the one or more medication removal parameters includes one or more of a quantity of expiry removal attempts from the ADC, a quantity of expiry removal attempts for which no expired items were removed from the ADC, a quantity of expiry removal attempts for which a corresponding pocket of the ADC was emptied, a beginning count of the medication in pockets of the ADC, an end count of the medication in the pockets of the ADC, and a quantity of the medication in the pockets of the ADC.

In some aspects, the method may include determining, based on the plurality of transaction records, a type of medical professional performing the transaction.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the management of medication delivery trips between a pharmacy and a dispensing cabinet at a medical facility, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a block diagram illustrating a trip management system, in accordance with some example implementations;

FIG. 2 depicts a system diagram illustrating an example of a trip management system, in accordance with some example implementations;

FIG. 3A depicts an example representation of transaction data, in accordance with some example implementations;

FIG. 3B depicts an example representation of trip data that can be generated based on transaction data, in accordance with some example implementations;

FIG. 4A depicts an example visualization of at least one trip metric, in accordance with some example implementations;

FIG. 4B depicts an example visualization of at least one trip metric, in accordance with some example implementations;

FIG. 5A depicts an example visualization of at least one trip metric, in accordance with some example implementations;

FIG. 5B depicts an example visualization of at least one trip metric, in accordance with some example implementations;

FIG. 5C depicts an example visualization of at least one trip metric, in accordance with some example implementations;

FIG. 5D depicts an example visualization of at least one trip metric, in accordance with some example implementations;

FIG. 6A depicts an example visualization of a transaction type trip metric, in accordance with some example implementations;

FIG. 6B depicts an example visualization of a transaction type trip metric, in accordance with some example implementations;

FIG. 6C depicts an example visualization of a transaction type trip metric, in accordance with some example implementations;

FIG. 7 depicts a flowchart illustrating a process for managing a medical workflow including trips from a pharmacy, in accordance with some example implementations; and

FIG. 8 depicts a block diagram illustrating a computing system, in accordance with some example implementations.

When practical, similar reference numbers denote similar structures, features, or elements.

### DETAILED DESCRIPTION

As part of their daily routine, medical professionals, such as pharmacy couriers, make trips from pharmacies to patient care areas to perform one or more transactions at one or more ADCs, which are automated dispensing devices that deliver medications to nurses or other caregivers as they are needed to medicate patients. Unique medications are stored in pockets of the ADCs, whose inventory quantities are governed by minimum and maximum values that, among other things, stimulate requests for refill. To maintain the ADCs, couriers may take one or more trips from a pharmacy to the ADCs to perform one or more maintenance transactions at the ADCs, and return to the pharmacy. For example, a transaction may include adding a new medication to an ADC (e.g., loading), removing medication that is no longer used from the ADC (e.g., unloading), removing medication from one ADC and transferring the medication to another ADC (e.g., destocking), taking inventory of pockets in the ADC (e.g., inventory), refilling a pocket of the ADC (e.g., restocking), emptying a return bin of the ADC, removing expired medications from the ADC, and/or the like. Some medical facilities generate tens or hundreds of thousands of transaction records per month when transactions are performed at the ADCs.

The trips taken by the medical professionals to perform such transactions may be planned or scheduled, or may be unplanned or ad hoc. For example, pharmacies may have one or more scheduled delivery trips from the pharmacies to the ADCs that occur on or about the same time every day, have a longer duration, include a wider array of transaction types and number of transactions, and include visiting a large number of ADCs. In some instances, all or a portion of these scheduled trips may be inefficient, redundant, and/or unnecessary. For example, couriers may take poorly planned routes to perform the transactions at each ADC during a trip, potentially expiring medications stored at the ADC may no longer be present in the ADC at the time of the scheduled trip, and/or the like.

Often, couriers make ad hoc trips from the pharmacies to particular ADCs to meet the needs of a patient care area served by the particular ADCs. These trips may be relatively short in duration, involve visiting only a small number of ADCs, involve performing a small number of transactions, and involve performing a small number of transaction types at the ADCs. However, the ad hoc trips may be time consuming, may interfere with other tasks performed by couriers, and may interfere with the administration of medications to patients.

There is currently no mechanism by which the pharmacy accounts for such ad hoc trips, so these trips are undocumented except for the transactional data (e.g., the transaction records) they generate at the ADC. Indeed, even scheduled trips do not generate records other than the transaction records generated at the ADC as various actions are performed at the stations. The trip management system consistent with implementations of the current subject matter may help to systematically identify and characterize trips based on the transaction data to maximize the value of the scheduled trips, to minimize the number of ad hoc trips, and to distinguish between the types of trips for more efficient planning. This helps to determine efficient medical workflows, trip planning, and route planning during trips, saving time, resources, and expense for the pharmacy and/or medical facility, while improving the ability for caregivers to provide better care for patients.

In some instances, information relating to the individual trips, such as the type of trip (e.g., scheduled or ad hoc), when the trips are taken, the duration of the trip, the transactions performed during the trip, and the types of transactions performed during the trip, may be helpful in maximizing the efficiency of medical workflows and trip planning, and measuring the impact of improvements in medication inventory optimization. However, it may be difficult to distinguish between the type of trip taken by couriers each day, since the type of trip taken is not generally logged by the couriers and both scheduled and ad hoc trips may occur at or near the same time. The trip management system consistent with implementations of the current subject matter may implement an analytics engine, which may apply one or more statistical analysis techniques and/or machine learning models trained to distinguish and identify between scheduled and ad hoc trips, and/or determine the approximate times at which scheduled trips occur. Thus, the trip management system consistent with implementations of the current subject matter may provide better information for improving trip planning and medical workflow efficiency.

For example, the trip management system may, in real time or near real time, identify that a trip from the pharmacy to one or more ADCs has started. Once the trip is identified, the trip management system may adjust one or more ADCs expected to be visited on that trip in anticipation of a visit. In some implementations, the trip may further be characterized as an ad hoc trip. In such instances, the adjustment to the one or more ADCs may include activating additional surveillance at the ADC when visited by the courier performing the ad hoc trip. Another example adjustment may be to expedite transmission of a notification sent by the ADC indicating completion of the visit by the courier because an ad hoc trip may be associated with a critical (e.g., “stat”) medication needed for a patient.

The trip management system may annotate and store the trip and trip type information in association with the transaction records generated at the ADC. In this way, the trip management system provides valuable annotations to the transaction data that is available to other hospital information systems. For example, the trip information may be used by a diversion detection system. The diversion detection system may assess when trips are occurring and which couriers are performing the trips. These signals may be useful in identifying patterns that may be consistent with diversion activity. In some systems, the trip type may be used to prioritize resources for diversion analysis such as by reviewing transactions associated with an ad hoc trip before a scheduled trip. In some implementations, the trip type may be used to weight a transaction's emphasis as part of diversion detection. For example, a longer time between visits on an ad hoc trip may be more suspicious than during a scheduled trip. In this case, any value used by a diversion detection algorithm from an ad hoc trip may be given more emphasis than a value from a transaction associated with a scheduled trip.

The annotations may also be used to model future scheduling. For example, the system may identify a scheduled trip that is often accompanied by an ad hoc trip 10 minutes (or another amount of time) later. The system may then provide a suggestion to adjust the scheduled trip by 10 minutes to obviate the need for the ad hoc trip.

As another example, once the system has properly annotated trips, a success metric (also referred to herein as a “trip metric”) for each trip can be generated. For example, if the trip was taken to refill a medication, the amount of medication needed to refill a container may drastically change between the time the courier leaves the pharmacy and arrives at the container. This problem is sometimes referred to as “underfill.” The system may compare trip data to inventory levels. The success of a refill visit may be assessed using a par level (e.g., desired inventory level for a container) and the actual level in the container. The success metric may identify how closely the desired inventory level corresponds to the actual inventory level.

For example, in some instances, trips, including both scheduled and ad hoc trips, to maintain the ADCs, may be poorly planned. As noted above, ADCs may include drawers having pockets, which contain a particular medication. Each pocket has established par levels (e.g., minimum and maximum quantities) that are determined based on the frequency of use for that particular medication for the particular patient population served by that ADC. When the inventory of the pocket falls below the minimum par level, the ADC may issue a “critical low” warning and prompt for a refill to occur. Based on the information at hand, a medical professional, such as the pharmacy courier, prepares for a delivery, acquires the amount of medication needed to replenish the pocket to its maximum par level, assembles the supplies to be delivered to the ADC, and takes a scheduled trip, which may take as long as 2-3 hours or longer. In some cases, the pocket may already be depleted. In such cases, the ADC may issue a stock-out warning to the pharmacy, resulting in an ad hoc trip to replace the medication that is no longer available at the particular ADC.

Generally, the pockets of the ADCs have a fixed and known geometry, limiting the inventory that can be contained within the pockets. ADCs may also have physical limits on the number of pockets that can be contained within the ADCs. Thus, increasing the maximum par level or increasing the number of pockets contained within the ADCs may not help to maintain a sufficient level of medication stored within the ADCs. Additionally, systems may not allow for efficiently restocking the ADCs to be evaluated, may not allow for a determination of the time and labor expense associated with trips to ADCs to refill or restock the ADCs, may not allow for pharmacy workers who receive the stock-out warning while a scheduled delivery is in progress to determine whether the medication has already been supplied to the ADC or will be supplied by the scheduled delivery in progress or another scheduled delivery, and/or the like. The trip management system consistent with implementations of the current subject matter annotates trip and trip type information, generates at least one trip metric and generates and/or displays visualizations of past, and currently in-progress trips. The visualizations may help couriers determine whether an ad hoc trip is required to refill or restock the ADC, and/or the trip management system may (e.g., via the analytics engine) determine whether and/or when an ad hoc trip is required to refill or restock the ADC.

The trip management system consistent with implementations of the current subject matter may additionally or alternatively schedule and/or suggest a schedule for stocking or refilling the medications at the ADCs. Thus, the trip management system described herein may help to reduce the number of unnecessary ad hoc trips taken by couriers, may help to prevent or limit stock-outs, and/or may help to more efficiently restock or refill ADCs, while maintaining a sufficient level of the medications contained within the pockets of the ADCs. This helps prevent the delivery of medication to the ADC from interfering with dispensing medications from the ADCs to treat patients. Such configurations may also help to better time the trips to ensure that the ADCs are sufficiently restocked and/or refilled to account for times when there is a high demand on the medication dispensed by the ADC to treat the patients.

In some instances, consistent with implementations of the current subject matter, a pocket of an ADC is manually assigned an expiration date based on the earliest expiration date associated with medications stored in that pocket. Each time a pocket of the ADC is refilled, the medical professional generally reviews the contents of the pocket and, if needed, enters a new expiration date for the pocket based on the earliest expiration date among the medications contained in the pocket. Since caregivers generally do not retrieve medications from the pocket of the ADC in any particular order, it is possible that all the medications that might have expired since the last refill of the pocket of the ADC have been removed. As a result, up to 50% or more of the transactions performed at the ADC to remove potentially expiring medication from the ADC may result in the medical professional not finding any expired medication in the pocket of the ADC. Thus, in a large number of cases, resources of the pharmacy and/or medical facility are wasted, as the medical professional did not need to visit the ADC during a trip, did not need to take a scheduled or ad hoc trip to the ADC, and/or did not need to perform the transaction at the ADC. The trip management system consistent with implementations of the current subject matter (such as via the analytics engine) generates and displays a visualization of at least one trip metric to determine and/or enable determination as to whether expired medications are likely to be contained within each pocket of each ADC, and from which pockets, the expired medications should be removed.

Accordingly, the trip management system may allow for better trip planning during a medical workflow and for a reduction in the number of ad hoc trips to a reduced number of ADCs with a reduced number of transactions performed at the ADCs. The trip management system may additionally or alternatively allow for more efficient scheduling of restocking and refilling ADCs, removing of expired medications from the ADCs, and/or the like. Additionally or alternatively, the trip management system may generate and display a visualization of one or more trip metrics to allow for and/or suggest more efficient medical workflows.

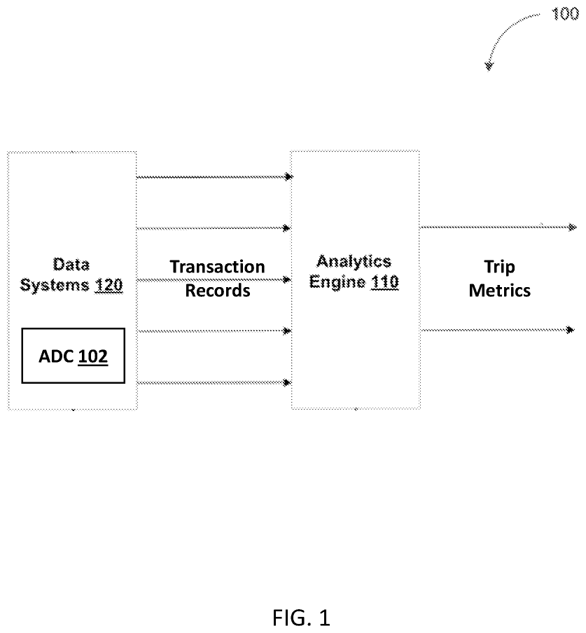

FIG. 1 depicts a system diagram illustrating a trip management system 100, in accordance with some example implementations. Referring to FIG. 1, the trip management system 100 may include an analytics engine 110 that is communicatively coupled with one or more data systems 120. The analytics engine 110 may analyze data received from the one or more data systems 120, such as data collected and/or determined during a medical workflow including one or more trips. The analytics engine 110 may then apply one or more statistical analysis techniques and/or machine learning models to the received data to identify distinct trips, predict whether a trip is a scheduled trip or an unscheduled (e.g., ad hoc) trip, or generate at least one trip metric characterizing at least one aspect of one or more trips. Based on the input data and/or the generated at least one trip metric, the analytics engine 110 may generate and/or display, such as via a client device and/or a handheld device, a visualization that indicates an effectiveness of the trips, and allows for more efficient planning of one or more aspects of the medical workflow, such as the one or more trips.

For example, a user interface of a client device (e.g., the client device 250) and/or a handheld device may present an initial representation or visualization of individual transactions along with a control element that, when activated, causes the trip management system to generate and/or present a visualization or representation of trips. The representation of trips may be based on the trip metric or other assessment of the individual transaction data as described herein. The user interface may include a subsequent control element that, when activated, causes the trip management system to present a visualization or representation that differentiates trips based on trip type (e.g., scheduled, ad hoc). The representations may be provided as schedules, including time or date information. The analytics engine 110 may also provide recommendations to identify schedule changes that could avoid or limit ad hoc trips and/or to prevent underfill for scheduled trips.

The analytics engine 110 may additionally or alternatively apply one or more statistical analysis techniques and/or machine learning models to the received data, and/or use the generated at least one trip metric to trigger an investigative workflow, suggest an updated medical workflow, schedule at least a portion of the medical workflow, distinguish between a scheduled trip and an ad hoc trip, distinguish between different types of medical professionals participating in the medical workflow, detect a diversion of medication during a medical workflow, and/or the like.

In some implementations, each trip of the one or more trips of the medical workflow may include a sequence of inventory management transactions performed by a medical professional, such as a pharmacy courier or worker, at one or more dispensing cabinets, such as the one or more ADCs. The ADCs may each include one or more pockets or other enclosures that contain a medication to be dispensed to a caregiver or clinician for treating a patient. An example dispensing cabinet is described in U.S. Pat. No. 8,195,328, which is commonly owned and hereby incorporated by reference in its entirety. The ADCs may be located in one or more patient care areas. As used herein, a care area may refer to an area in a specific medical facility that is designated based on function and/or location. For instance, a facility such as a hospital may include a plurality of care areas such as, for example, for medical-surgical, intensive care, emergency, observation, non-acute, infusion center, outpatient, and/or the like.

Each trip of the one or more trips may be taken by a medical professional from a pharmacy to an ADC, from an ADC to another ADC, and/or from an ADC to the pharmacy. In some instances, the ADC may be located at a different clinic or campus than the pharmacy. The one or more trips may also include one or more scheduled trips and/or ad hoc trips. The inventory management transactions (also referred to herein as a “transaction” or “transactions”) performed during the one or more trips may include a refill (e.g., refilling one or more pockets of the ADC with a medication when the medication was previously assigned to that pocket), a refill stock-out (e.g., refilling one or more pockets of the ADC that are out of stock of the medication), a refill-critical low (e.g., refilling one or more pockets of the ADC that contain an amount of a medication that is less than or equal to a threshold level), a refill-less than max (e.g., refilling the ADC to less than a maximum amount of the medication), an inventory (e.g., counting an amount of a medication in each pocket of the ADC), a load (e.g., loading one or more pockets of the ADC with medication when the medication is assigned to a previously unassigned pocket and then placed in that pocket), an unload (e.g., removing medication, such as medication that is no longer used, from one or more pockets of the ADC and the pocket becomes unassigned), an expiry check (e.g., checking an expiration date of the medication contained within one or more pockets of the ADC), an expiry removal (e.g., removing expired medication from one or more pockets of the ADC), a destock (e.g., removing medication from one or more pockets, where the one or more pockets remain assigned to the medication), emptying a return bin of the ADC, and/or the like.

Referring to FIG. 1, the one or more data systems 120 may include medical devices such as, for example, the dispensing cabinets, such as the ADCs (ADCs 102), infusion pumps, wasting stations, and/or the like. As shown in FIG. 1, the one or more data systems 120 may collect data that arise from medical professionals, such as pharmacy couriers, clinicians, and/or the like, interacting with the one or more data systems 120 during one or more transactions, as described herein. For instance, each of the one or more data systems 120 may be configured to generate a transaction record in response to and/or during a transaction. For example, a medical professional engaging in a transaction at an ADC during the one or more trips may trigger the generation of one or more transaction records. The one or more transaction records may be determined as a result of an input received by the ADC, a calculation or determination made by the ADC, a medical professional identification tag scanned by the ADC (e.g., via RFID), and/or the like. Accordingly, the analytics engine 110 may receive, from the one or more data systems 120, a plurality of input data including the one or more transaction records. It should be appreciated that the input data from the one or more data systems 120 may include at least a portion of the one or more transaction records that are generated in response to interactions between the ADCs with one or more different couriers.

In some implementations, the one or more transaction records may include one or more transaction values corresponding to a timestamp, a login time at an ADC, a logout time at the ADC, a patient identifier, a device identifier, a medical professional identifier, a medication identifier, a prescription order identifier, an inventory information, a patient status, a shift identifier, a location tracking identifier, an infusion information, a compounding information, an administration information, a working off clock indicator, an electronic health record identifier, and/or the like. Additionally or alternatively, the one or more transaction records may include one or more transaction values corresponding to a start time of the transaction and/or a stop or completion time the transaction. Additionally or alternatively, the one or more transaction records may include a quantity of transactions performed at each ADC during a period of time, such as during a trip, a quantity of ADCs visited within a period of time, an identity of each ADC visited during a period of time, an identity of each type of transaction performed at each ADC, one or more refill parameters, one or more medication removal parameters, and/or the like. In some implementations, the one or more refill parameters may include a number of refills, a number of refills to pockets of the ADC that are empty, a number of refills to pockets of the ADC that are below a minimum PAR level, a number of refills that do not completely refill a corresponding pocket of the ADC to a maximum PAR level, a beginning count of the medication in the pockets of the ADC, an end count of the medication in the pockets of the ADC, a quantity of the medication in the pockets of the ADC, and/or the like. In some implementations, the one or more medication removal parameters includes a quantity of expiry removal attempts from the ADC, a quantity of expiry removal attempts for which no expired items were removed from the ADC, a quantity of expiry removal attempts for which a corresponding pocket of the ADC was emptied, a beginning count of the medication in pockets of the ADC, an end count of the medication in the pockets of the ADC, a quantity of the medication in the pockets of the ADC, and/or the like. At least the time stamp, the period of time, the start time, the stop time, and/or the like may be included in at least a portion of time information (described in more detail below).

In some implementations, the one or more transaction records includes an elapsed time (which may also be referred to herein as an “elapsed trip time”). In other implementations, the elapsed time may be determined based on at least the time information. The elapsed time may include an amount of time that elapsed since the start time of a particular transaction. In other words the elapsed time may include a length of the period of time for the courier to perform the trip (e.g., leave the pharmacy, perform a transaction at one ADC, and return to the pharmacy). In some implementations, the elapsed time is determined based at least in part on a comparison of the time information (e.g., a comparison of a start time and/or stop time). The elapsed time may be an amount of time elapsed between sequential transactions at different ADCs. The elapsed time may include an amount of time that elapsed between two adjacent start times. In some implementations, the elapsed time is a length of a particular transaction. The elapsed time may be determined based on a start time and/or an end time of the particular transaction using the timestamp of the login time and/or the logout time at the ADC.

It should be appreciated that the one or more data systems 120 may also provide non-transactional data to the analytics engine 110. For instance, the one or more data systems 120 may provide electronic medical records (EMRs), which may include a patient's history including, for example, past opioid use and/or the like. While the act of creating, updating, and/or retrieving an electronic medical record may generate a timestamp transaction record, the electronic medical record itself is not a transaction record. In some implementations, the elapsed time may be determined based at least in part on the non-transactional data.

As noted above, a medical facility may generate thousands to tens of thousands or more of transaction records each day. However, couriers often do not document when they begin or end their trips from the pharmacy, to one or more ADCs, and/or back to the pharmacy. As a result, it is difficult to track which transaction records are part of a particular trip and whether a trip is a scheduled trip or an unscheduled or ad hoc trip. Thus, it is difficult to improve trip scheduling and efficiency of medical workflows at a medical facility.

In some implementations, the non-transactional data additionally or alternatively includes a timestamp of a courier exiting and/or entering the pharmacy. While the couriers may not generally document the start or end of their trips, pharmacies and/or medical facilities may include one or more features that track a possible start and/or end time of a trip. The one or more features may provide entry and/or exit data related to at least the pharmacy that can improve the accuracy of trip detection and the determination of a length of time of the trip, such as the elapsed time as described herein. For example, a pharmacy or another portion of the medical facility may include a lock, such as a pharmacy lock, and/or another tracker. The lock may track a timestamp and/or an identifier associated with a key card of a courier. For example, the lock may include recording a swipe of a key card of the courier. The analytics engine 110 may compare consecutively recorded swipes by the same courier (e.g., based on the identifier of the courier). The lock may additionally and/or alternatively track a Bluetooth connection or other wireless signal of the courier entering and/or exiting the pharmacy. The analytics engine 110 may use the recorded information to identify a trip and/or determine a start and/or end time of a particular trip.

Consistent with implementations of the current subject matter, the analytics engine 110 may receive, as an input, time information such as the start time and/or the end time (or another one of or combination of the transaction records described herein) of one or more transactions, and determine, based on the time information including the start time and/or the end time (or another one of or combination of the transaction records described herein) of the one or more transactions, whether a portion of the transaction records represents a unique trip. In some implementations, the transaction records of the transactions performed at the ADCs may be grouped by a courier or other medical professional and/or sorted in an ascending time sequence. This may help to determine the portion of sequential transactions and corresponding transaction records forms a part of or the entirety of the unique trip, since as noted above, couriers often do not document the time at which they engage in a trip (e.g., when the courier leaves and/or returns to the pharmacy).

As an example, the analytics engine 110 may compare one or more of the transaction records with a threshold to determine whether a group of transactions form a part of or the entirety of a trip. For example, the analytics engine 110 may identify, based at least in part on one or more of (i) a comparison of time information (e.g., the start time, the end time, a total time, or the like of a transaction) of or between respective transactions and a threshold inter-transaction trip time and (ii) a comparison of an elapsed time for a portion of the transactions and a threshold elapsed time, a sequence of the portion of the group of transactions as a unique trip.

The threshold, such as the threshold inter-transaction trip time, the threshold elapsed time, a threshold inter-station time, and the like, may be predetermined and/or may be dynamically determined by the analytics engine 110. In some implementations, the threshold may be determined based on historical trip data and/or one or more characteristics of the medical facility, such as a size of the medical facility, a number of patient rooms at the medical facility, a number of ADCs at the medical facility, a distance from a pharmacy to a farthest possible location of a transaction of the plurality of transactions, a square footage of the medical facility, a number of patients being treated at the medical facility, a number of elevators at the medical facility, and a number of floors at the medical facility. In some implementations, a larger medical facility may have a larger threshold than a smaller facility, since the expected round trip time for a trip would be longer. The threshold may be 10 to 30 minutes, 30 to 60 minutes, 60 to 90 minutes, 90 to 120 minutes, longer, or other ranges therebetween. In some implementations, the threshold may be determined by a machine learning model trained to determine the threshold based on one or more of a longest period of time between transactions, the shortest period of time between transactions, the geography of the medical facility, the transit mechanisms between the pharmacy and the patient care areas, and/or the like.

Based on the identification of the portion of the transactions (or transaction records) as a unique trip, the analytics engine 110 may annotate one or more transaction records with trip information generated using at least some of the features described. The annotation may include a trip identifier associating several transactions (or transaction records) as part of the unique trip. The annotation may include a trip type identifier to indicate the type of trip which has been identified (e.g., ad hoc, scheduled, or undetermined).

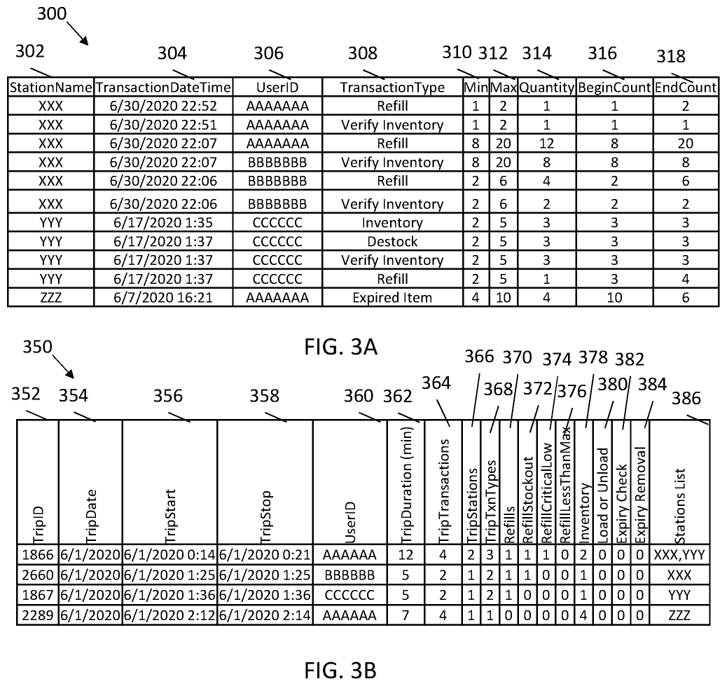

Consistent with implementations of the current subject matter, the analytics engine 110 may generate one or more data representations that include the one or more transaction records. The one or more representations may be generated based on a series of transaction records associated with one or more medical workflows including one or more trips performed by one or more couriers. For example, FIG. 3A illustrates a first representation 300 of one or more transaction records. As shown in FIG. 3A, the one or more transaction records includes an ADC name 302, a timestamp 304 of the transaction performed at the ADC, an identifier 306 of the medical professional performing the transaction at the ADC, a transaction type 308 of the transaction performed at the ADC, a minimum quantity 310 of a medication that should be contained within the ADC, a maximum quantity 312 of a medication that should be contained within the ADC, a quantity 314 of a medication that is contained within the ADC, a beginning count 316 of the medication contained within the ADC during the transaction, and an end count 318 of the medication contained within the ADC during the transaction.

FIG. 3B illustrates a second representation 350 including one or more values associated with predicted or identified trips. As shown in FIG. 3B, the trip records includes an identifier 352 of a trip, a date 354 of the trip, a start time 356 of the trip, a stop time 358 of the trip, an identifier 360 of the medical professional taking the trip, a trip duration 362 of the trip, a quantity 364 of trip transactions performed during the trip, a quantity 366 of ADCs visited during the trip, a quantity 368 of types of transactions performed during the trip, a quantity 370 of refill transactions, a quantity 372 of refill stock out transactions performed during the trip, a quantity 374 of refill critical low transactions performed during the trip, a quantity 376 of refills less than maximum transactions performed during the trip, a quantity 378 of inventory transactions performed during the trip, a quantity 380 of load or unload transactions performed during the trip, a quantity 382 of expiry check transactions performed during the trip, a quantity 384 of expiry removal transactions performed during the trip, and a list 386 of ADCs visited during the trip. In some implementations, the trip identifier 352 may be associated with an individual transaction to provide an annotation indicating which trip the system associated the transaction with. The associations may be reviewed using a graphical user interface of the client device and/or handheld device. The user interface may include a control element to re-associate a transaction with a different trip. The re-association may be used by the system to retrain the model of the analytics engine 110 for future trip identification.

As noted above, the analytics engine 110 generates, based on the input data including the one or more transaction records and/or trip records, at least one trip metric (e.g., one, two, three, four, five, six, seven, eight, nine, ten, or more trip metrics). The at least one trip metric may be indicative of a characteristic of each of the one or more trips, such as an effectiveness of the trip or a series of trips, and may allow for more efficient planning of a medical workflow and/or a reduction in a number of ad hoc trips that are part of the medical workflow. In some implementations, the at least one trip metric includes a duration of at least one trip, a quantity of ADCs visited during at least one trip, a quantity of a transaction type of at least one transaction performed at the ADC during a trip, a quantity of different transaction types of the transactions performed at the ADC during at least one trip, a total quantity of trips in the medical workflow, a total quantity of refill transactions performed during the medical workflow, a total quantity of critical low refill transactions performed during the medical workflow, a total quantity of refills less than maximum transactions performed during the medical workflow, a total quantity of inventory transactions performed during the medical workflow, a total quantity of load and/or unload transactions performed during the medical workflow, a total quantity of expiration no-remove transactions performed during the medical workflow, a total quantity of expiration remove transactions performed during the medical workflow, a total duration of the medical workflow, a total quantity of transactions performed during the medical workflow, a total quantity of stock out refills transactions performed during the medical workflow, and/or the like. In some implementations, the at least one trip metric includes a generated statistic that indicates an effectiveness of the one or more trips.

In some implementations, based on the generated at least one trip metric, the analytics engine 110 may generate and/or display, such as via a client device and/or a handheld device, one or more visualizations, which may indicate an effectiveness of the medical workflow. In some implementations, the one or more visualizations may allow for the medical workflow to be updated and/or the planning of the one or more trips of the medical workflow to be made more efficient, by, for example, reducing a number of ad hoc trips within the medical workflow. The visualizations may illustrate a quantity of each at least one metric over time, which provides useful information to track and/or update medical workflows performed at a medical facility.

The visualization may additionally or alternatively allow selection of a particular class of transactions for a trip and, after selection, present a listing of the transactions of the selected class within the trip. For example, in some implementations, the trip management system 100 (e.g., the analytics engine 110) may cause presentation of a first plot of trips representing a plurality of trips at the medical facility. The first plot of trips may be presented in response to the selection of the particular class of transactions for a trip, and/or a selection of a particular class of trips. The system 100 may receive a message requesting a second plot of trips showing at least two trip types (e.g., scheduled, unscheduled, undetermined, etc.) for the medical facility. The system 100 may cause presentation of the second plot of trips. The second plot of trips may include a first visualization for at least one trip identified as a first trip type and a second visualization for at least one trip identified as a second trip type.

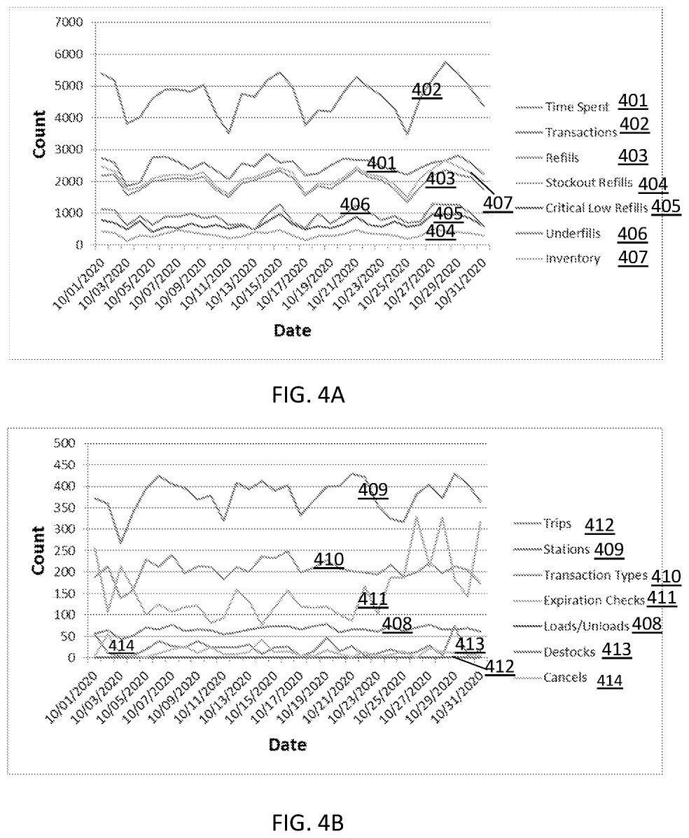

FIGS. 4A and 4B illustrate example visualizations, consistent with implementations of the current subject matter. The visualizations illustrated in FIGS. 4A and 4B each show a plurality of trip metrics in relation to one another. The plurality of trip metrics illustrated in the visualizations were generated based on one or more transaction records determined for trips taken each day over a month. The visualizations show a frequency of each transaction type over the captured month. For example, FIG. 4A illustrates a visualization generated based on a plurality of trip metrics including a time spent during a transaction 401, a quantity of transactions performed 402, a quantity of refill transactions performed 403, a quantity of stockout refill transactions performed 404, a quantity of critical low refill transactions performed 405, a quantity of underfill transactions performed 406, and a quantity of inventory transactions performed 407. FIG. 4B illustrates a visualization generated based on a plurality of trip metrics including a quantity of trips taken 408, a quantity of ADCs visited 409, a quantity of different transaction types performed 410, a quantity of expiration check transactions performed 411, a quantity of load/unload transactions performed 412, a quantity of destock transactions performed 413, and a quantity of cancel transactions performed 414.

In some implementations, based on the generated visualizations, the analytics engine 110 may trigger an investigative workflow, such as when the analytics engine 110 determines that a spike occurred in at least one of the generated trip metrics. The analytics engine 110 may determine that a spike occurred in at least one of the generated trip metrics when a value of the generated trip metric is greater than or equal to a threshold value. Additionally or alternatively, the analytics engine 110 may determine that a spike occurred in at least one of the generated trip metrics when a value of the generated trip metric for a period of time (e.g., one day) is higher than a value of the generated trip metric of another period of time (e.g., another day) and/or an average value of the generated trip metric over a period of time, by a threshold amount (e.g., 1 to 10% higher, 10 to 20% higher, 20 to 30% higher, 30 to 40% higher, 40 to 50% higher, and/or the like). As an example, the visualization shown in FIG. 4B shows a spike in the cancel transactions performed 414 on or about Oct. 3, 2020.

In some implementations, when the analytics engine 110 determines that a spike in the value of the generated trip metric has occurred, such as when the value of at least one trip metric is greater than or equal to a threshold value, the analytics engine 110 may trigger an investigative workflow. The investigative workflow may include generating and sending an alert. Alternatively and/or additionally, the investigative workflow may include the analytics engine 110 configuring the one or more data systems 120 to activate one or more surveillance devices (e.g., video cameras, still image cameras, audio recorders, and/or the like) at a medical management device (e.g., an ADC, a wasting station, and/or the like), such as whenever a medical professional interacts with one or more management devices, such as the ADCs. In some implementations, the investigative workflow may further include the analytics engine 110 configuring the one or more data systems 120 to isolate medication stored within one or more management devices, such as the ADCs. In some implementations, the one or more ADCs may include an ADC accessed by a particular medical professional.

Additionally or alternatively, the analytics engine 110 may trigger the investigative workflow by at least activating a sensor coupled to one or more ADCs. The activation may include transmitting a control message to the sensor and/or the ADCs to configure the sensor and/or the ADCs to initiate collection of information for the investigative workflow, such as biometrics of the target of the investigation, electronic medical records for patients interacted with by or near the target of the investigation, location information for the target during one or more trips, and/or the like. This can be particularly useful to collect information without necessarily alerting a target medical professional of the investigation. If the target medical professional of the investigation were made aware of the data collection, the target of the investigation may alter behavior or take further steps to avoid detection or undermine the integrity of the investigation. Such features provide a technical solution to ensure efficiency and reliability of the investigative workflow.

Additionally or alternatively, the investigative workflow may be performed, such as by the analytics engine 110, to develop an updated and/or more efficient medical workflow and/or to track an effectiveness of the medical workflow. As an example, if the analytics engine 110 detects a spike in a quantity of critical low refills, the analytics engine 110 may determine that the ADCs accessed during the one or more trips may be improperly restocked and/or refilled and/or may be restocked and/or refilled at an incorrect time. In such instances, the analytics engine 110 may suggest an updated medical workflow in which the ADCs are properly restocked and/or refilled to reduce a number of required ad hoc trips during the medical workflow, making the medical workflow more efficient.

In some implementations, based on the generated trip metrics and/or the visualizations, the analytics engine 110 may determine whether a trip of the medical workflow is a scheduled trip or an ad hoc trip. For example, the analytics engine 110 may apply one or more statistical model techniques and/or trained machine learning models to classify a trip as a scheduled trip and an ad hoc trip. Distinguishing between a scheduled trip and an ad hoc trip allows for a medical workflow to be updated (such as by the analytics engine 110) to reduce a number of ad hoc trips taken during the medical workflow. Doing so increases the efficiency of the medical workflow and improves the planning of the trips taken during the medical workflow. As an example, generation of the at least one trip metric and/or the visualization includes applying a machine-learning model trained to identify an approximate time at which the scheduled trip occurred, and identifying, using the machine-learning model, the approximate time at which the scheduled trip occurred.

In some implementations, the analytics engine 110 may determine, based on the generated trip metrics and/or the generated visualizations that the at least one trip is the scheduled trip or the ad-hoc trip. For example, the analytics engine 110 may determine that the at least one trip is the scheduled trip when the at least one trip occurs at regular times over two or more days, a length of time of the trip is greater than or equal to a threshold time (e.g., fifty minutes, 30 to 40 minutes, 40 to 50 minutes, 50 to 60 minutes, 60 to 70 minutes, and/or the like), a quantity of ADCs visited during the at least one trip is greater than or equal to a threshold quantity (e.g., 10 ADCs, 7 to 8 ADCs, 8 to 9 ADCs, 9 to 10 ADCs, 10 to 11 ADCs, 11 to 12 ADCs, and/or the like), a quantity of transactions performed during the at least one trip is greater than or equal to a threshold quantity (e.g., 22 transactions, 10 to 15 transactions, 15 to 20 transactions, 20 to 25 transactions, 25 to 30 transactions, 30 to 35 transactions, and/or the like), and/or a quantity of different transaction types performed during the at least one trip is greater than or equal to a threshold quantity (e.g., 4 transaction types, 2 to 3 transaction types, 3 to 4 transaction types, 4 to 5 transaction types, 5 to 6 transaction types, 6 to 7 transaction types, and/or the like). Additionally or alternatively, the analytics engine 110 may determine that the at least one trip is the ad-hoc trip when the at least one trip occurs at irregular times over two or more days, a length of time of the trip is less than or equal to a threshold time (e.g., fifty minutes, 30 to 40 minutes, 40 to 50 minutes, 50 to 60 minutes, 60 to 70 minutes, and/or the like), a quantity of ADCs visited during the at least one trip is less than or equal to a threshold quantity (e.g., 10 ADCs, 7 to 8 ADCs, 8 to 9 ADCs, 9 to 10 ADCs, 10 to 11 ADCs, 11 to 12 ADCs, and/or the like), a quantity of transactions performed during the at least one trip is less than or equal to a threshold quantity (e.g., 22 transactions, 10 to 15 transactions, 15 to 20 transactions, 20 to 25 transactions, 25 to 30 transactions, 30 to 35 transactions, and/or the like), and/or a quantity of different transaction types performed during the at least one trip is less than or equal to a threshold quantity (e.g., 4 transaction types, 2 to 3 transaction types, 3 to 4 transaction types, 4 to 5 transaction types, 5 to 6 transaction types, 6 to 7 transaction types, and/or the like).

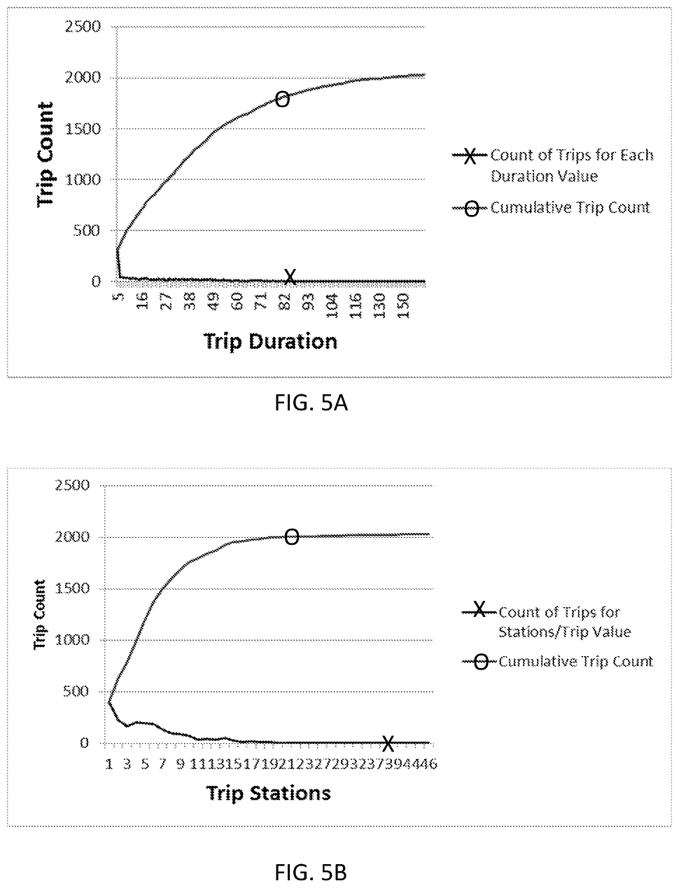

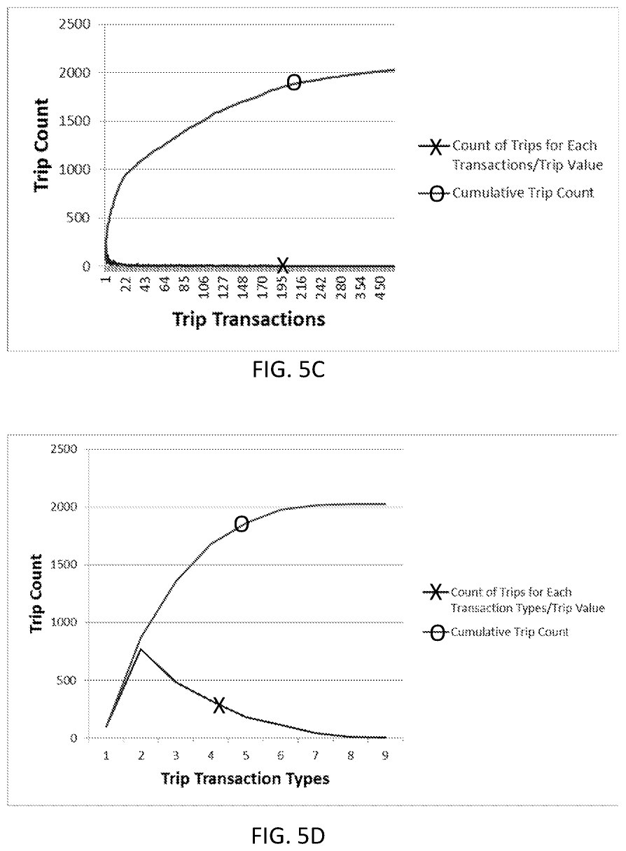

FIGS. 5A-5D illustrate visualizations generated, based on the one or more trip metrics, by the analytics engine 110, consistent with implementations of the current subject matter. In particular, FIGS. 5A-5D illustrate visualizations showing a relationship between a quantity of a particular type of trip metric (e.g., trip duration, ADCs visited, quantity of transactions, and transaction types per trip) and trip count. In other words, FIGS. 5A-5D illustrate visualizations showing the frequency of each generated trip metric. Based on the generated visualizations and/or the generated trip metrics, the analytics engine 110 may determine whether a trip is likely to be an ad hoc trip or a scheduled trip. For example, the analytics engine 110 may determine an inflection point within a visualization between trips that are likely ad hoc trips and trips that are likely scheduled trips. The inflection point may indicate a change in a slope of a curve (e.g., a cumulative trip count curve) that is greater than or equal to a threshold slope. In some implementations, the analytics engine 110 may determine that a trip is likely to be an ad hoc trip when the value of the trip metric is less than a threshold value. Alternatively, the analytics engine 110 may determine that a trip is likely to be a scheduled trip when the value of the trip metric is greater than or equal to a threshold value.

For example, FIG. 5A illustrates an example visualization showing a comparison between trip count and trip duration, consistent with implementations of the current subject matter. The duration of the trip may include a time difference between a first transaction and a last transaction in a sequence of transactions of the medical workflow in addition to an amount of time a medical professional takes to travel between a pharmacy and the first ADC during the medical workflow, and an amount of time a medical professional takes to travel between the last ADC and the pharmacy during the medical workflow. The maximum amount of time and/or the minimum amount of time between transactions, from the pharmacy to the first ADC, and/or from the last ADC (which may be the first ADC or another ADC) to the pharmacy, may change depending on the medical facility, as each medical facility has a different size and/or layout. Referring to FIG. 5A, the analytics engine 110 may determine that an inflection point occurs when the trip duration is approximately 50 minutes. This indicates that the trips having a trip duration of less than approximately 50 minutes are likely to be ad hoc trips. Thus, the analytics engine 110, such as via the visualization, may distinguish between the ad hoc trips and scheduled trips based on the trip duration trip metric.

As another example, FIG. 5B illustrates an example visualization showing a comparison between trip count and a quantity of ADCs visited, consistent with implementations of the current subject matter. In this example, the analytics engine 110 may determine that an inflection point occurs when the quantity of ADCs visited is approximately 10 ADCs per trip. This indicates that the trips having a quantity of ADCs visited of less than approximately 10 ADCs are likely to be ad hoc trips. Thus, the analytics engine 110, such as via the visualization, may distinguish between the ad hoc trips and scheduled trips based on the quantity of ADCs visited trip metric.

As a further example, FIG. 5C illustrates an example visualization showing a comparison between trip count and a quantity of transactions performed per trip, consistent with implementations of the current subject matter. In this example, the analytics engine 110 may determine that an inflection point occurs when the quantity of transactions performed per trip is approximately 22 transactions. This indicates that the trips having a quantity of transactions performed per trip of less than approximately 22 transactions are likely to be ad hoc trips. Thus, the analytics engine 110, such as via the visualization, may distinguish between the ad hoc trips and scheduled trips based on the quantity of transactions performed per trip metric.

As a further example, FIG. 5D illustrates an example visualization showing a comparison between trip count and a quantity of transaction types performed per trip, consistent with implementations of the current subject matter. In this example, the analytics engine 110 may determine that an inflection point occurs when the quantity of transaction types performed per trip is approximately 4 transaction types. This indicates that the trips having a quantity of transaction types performed per trip of less than approximately 4 transaction types are likely to be ad hoc trips. Thus, the analytics engine 110, such as via the visualization, may distinguish between the ad hoc trips and scheduled trips based on the quantity of transaction types performed per trip metric.

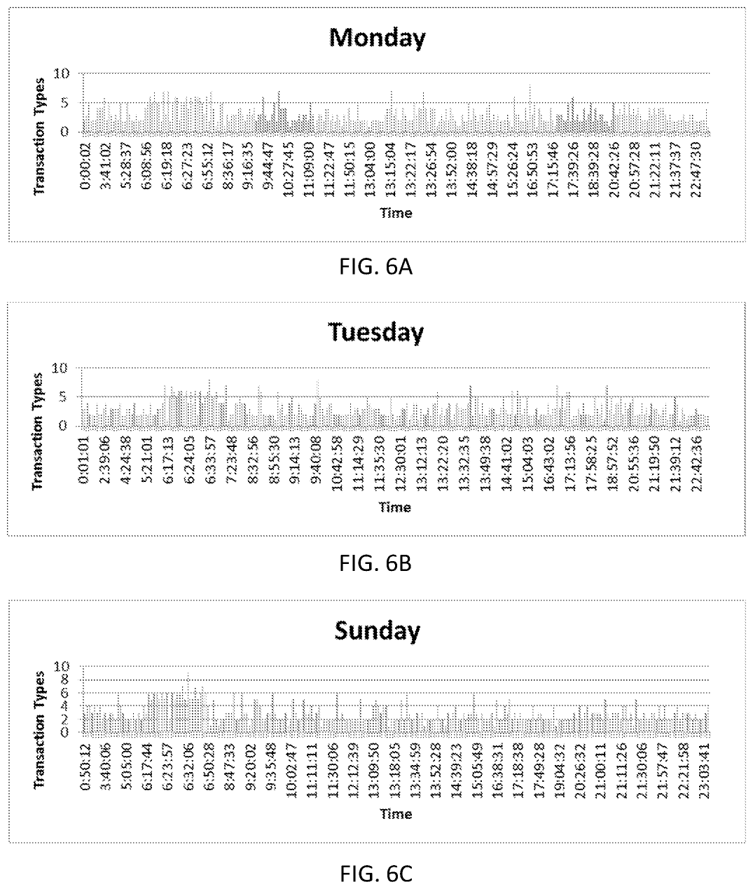

FIGS. 6A-6C illustrate additional visualizations generated by the analytics engine 110 based on the generated trip metrics, consistent with implementations of the current subject matter. For example, the generated visualizations shown in FIGS. 6A-6C illustrate a quantity of transaction types performed at various times over different days (e.g., FIG. 6A shows a visualization based on transaction records collected on a Monday, FIG. 6B shows a visualization based on transaction records collected on a Tuesday, and FIG. 6C shows a visualization based on transaction records collected on a Sunday). These visualizations allow for a comparison between trips conducted during different days, and highlight variances in the effectiveness of medical workflows performed during different days.

In some implementations, based on the generated trip metrics and/or the generated visualizations, the analytics engine 110 may suggest an updated medical workflow. The updated medical workflow may reduce a quantity of ad hoc trips from the plurality of trips of the medical workflow to improve the efficiency of the medical workflow. In some implementations, the updated medical workflow includes a sequence of the plurality of trips of the medical workflow, a time at which the plurality of trips should be performed, one or more transactions to perform during each of the plurality of trips, a route through the medical facility, and/or the like. Additionally or alternatively, the updated medical workflow may include a route for a robotic delivery system to perform a transaction at one or more ADCs during the medical workflow.

In some implementations, the analytics engine 110 schedules the updated medical workflow. For example, the analytics engine 110 may schedule at least one trip from the pharmacy to an ADC based on the generated trip metrics and/or the generated visualizations.

In some implementations, the generated metrics and/or the generated visualizations permit determination of an amount of time a medical professional spends on scheduled trips to better monitor performance of the medical professional during the one or more trips. Additionally or alternatively, the generated metrics and/or the generated visualizations permit determination of an amount of time the medical professional spends on ad hoc trips. This allows for the ad hoc trips to be enumerated and for the resource cost caused by the time spent on ad hoc trips.

Accordingly, because the analytics engine 110 monitors the medical workflow including the one or more trips and transactions performed during the trips, the analytics engine 110 allows for monitoring of the medication delivery process, and whether any changes made to the delivery process (such as via an updated medical workflow) have resulted in improvements in efficiency and in trip scheduling.

In some implementations, based on the generated metrics and/or the generated visualizations, the analytics engine 110 may identify a possible diversion of medication by a medical professional. For example, the analytics engine 110 may determine that a route taken by the medical professional during one or more trips is an anomalous route, indicating that a possible diversion has occurred. In some implementations, the analytics engine 110 determines that the route is an anomalous route when the route deviates from an average route by a threshold amount (e.g., 10 to 20%, 20 to 30%, 30 to 40%, 40 to 50%, and/or the like). Additionally or alternatively, the analytics engine 110 determines that the route is an anomalous route when one or more transactions and/or particular types of transactions occur at times when one or more transactions and/or particular types of transactions do not typically occur, and/or an ad hoc trip may generally not take place. Additionally or alternatively, the analytics engine 110 determines that the route is an anomalous route when one or more transactions and/or particular types of transactions occur for a length of time that is longer than an average amount of time and/or a threshold length of time (e.g., 15 minutes, 30 minutes, 45 minutes, one hour, two hours, three hours, or greater, or other ranges therebetween).

Additionally or alternatively, the analytics engine 110 determines that the route is an anomalous route when the analytics engine 110 based on a quantity of ADCs and/or patient care areas visited during a particular trip. For example, the analytics engine 110 may determine the route is anomalous when the quantity of ADCs and/or patient care areas visited during the particular trip meets a threshold quantity (e.g., 2, 3, 4, 5, 6, 7, 8, 9, 10, greater, or other ranges therebetween).

Additionally or alternatively, the analytics engine 110 may detect a possible diversion of medication and/or the route is an anomalous route based on a length of an identified trip. For example, based on the length of the identified trip meeting a threshold length (e.g., 15 minutes, 30 minutes, 45 minutes, one hour, two hours, three hours, or greater, or other ranges therebetween), the analytics engine 110 may detect the possible diversion of the medication and/or the route is an anomalous route.

Additionally and/or alternatively, the analytics engine 110 may detect a possible diversion of medication by the medical professional by at least tracking cycle counting at a medical management device (e.g., an ADC, a wasting station, and/or the like). For example, medical professionals are generally obligated to demonstrate reasonable levels of control over controlled substances, or other medications that are likely to be diverted. As part of the obligation, medical professionals may be required to cycle count at the ADC. In other words, the medical professionals may be required to verify an inventory of the medication stored at the ADC, such as at random time points. However, it some instances, it can be difficult to detect whether the inventory counts made by the medical professionals at the ADC are accurate and/or whether medical professionals at a particular medical facility are fulfilling their obligation to perform the cycle counting. The analytics engine 110 may help to determine whether a possible diversion has occurred, such as during cycle counting, and/or may help to detect whether it is likely that the medical professionals at the particular medical facility are fulfilling their obligation to perform the cycle counting.

The analytics engine 110 may receive, as an input (e.g., a transaction record), a total quantity of items counted at an ADC during a particular transaction at the ADC. Based on the total quantity of items counted at the ADC during the particular transaction, the analytics engine 110 may determine that the transaction at the ADC includes cycle counting. For example, the analytics engine 110 may determine the transaction include cycle counting when the total quantity of items meets a threshold quantity (e.g., 5, 10, 20, 30, 40, 50, greater, or other ranges therebetween). Thus, based on the transaction records generated during a transaction at the ADC, the analytics engine 110 may determine whether the medical professional is performing the required cycle counting at the ADC. Based on the total quantity of items counted and/or based on the determination the medical professional is performing cycle counting, the analytics engine 110 may determine that the medical professional is diverting the medication. For example, the analytics engine 110 may compare the total quantity of items counted during the transaction with an expected quantity of items. The analytics engine 110 may detect diversion of the medication, such as when the total quantity of items counted does not match the expected quantity of items.

In some implementations, the analytics engine 110 may provide useful information based on the determination that the medical professional is performing cycle counting, such as during a particular trip. For example, the analytics engine 110 may determine based on one or more transaction records (e.g., time stamps, or the like) generated during the cycle counting transaction and/or the determination that the medical professional has performed cycle counting, cycle counting data, including a length of time of the cycle counting, a length of time of the trip, a frequency of the cycle counting, or the like. Based on the cycle counting data, the analytics engine 110 may generate a visualization as described herein. The analytics engine 110 may additionally and/or alternatively indicate a monetary and/or a time burden on the medical facility associated with the cycle counting. In some implementations, when the analytics engine 110 determines that a possible diversion has occurred, the analytics engine 110 may trigger an investigative workflow. The investigative workflow may include generating and sending an alert. Alternatively and/or additionally, the investigative workflow may include the analytics engine 110 configuring the one or more data systems 120 to activate one or more surveillance devices (e.g., video cameras, still image cameras, audio recorders, and/or the like) at a medical management device (e.g., an ADC, a wasting station, and/or the like), such as whenever a medical professional suspected of diversion interacts with one or more management devices, such as the ADCs. In some implementations, the investigative workflow may further include the analytics engine 110 configuring the one or more data systems 120 to isolate medication stored within one or more management devices, such as the ADCs. In some implementations, the one or more ADCs may include an ADC accessed by the medical professional suspected of diversion.

Additionally or alternatively, the analytics engine 110 may trigger the investigative workflow by at least activating a sensor coupled to one or more ADCs. The activation may include transmitting a control message to the sensor and/or the ADCs to configure the sensor and/or the ADCs to initiate collection of one or more transaction records for the investigative workflow, such as biometrics of the target of the investigation, electronic medical records for patients interacted with by or near the target of the investigation, location information for the target during one or more trips, and/or the like. This can be particularly useful to collect information without necessarily alerting a target medical professional of the investigation. If the target medical professional of the investigation were made aware of the data collection, the target of the investigation may alter behavior or take further steps to avoid detection or undermine the integrity of the investigation. Such features provide a technical solution to ensure efficiency and reliability of the investigative workflow.

Consistent with implementations of the current subject matter, the analytics engine 110 may determine one or more thresholds as described herein. The one or more thresholds described herein may be determined using one or more statistical analysis techniques and/or trained machine learning models.

For example, the analytics engine 110 may receive as an input one or more parameters associated with the medical facility. The one or more parameters associated with the medical facility may include a size of the medical facility, a size of particular patient care areas within the medical facility, a distance between patient care areas within the medical facility, a distance between a pharmacy and a patient care area within the medical facility, a quantity of ADCs within the medical facility, or the like. Based on the input of the one or more parameters associated with the medical facility, the analytics engine 110 may output one or more thresholds used for the particular medical facility. Thus, trips taken and/or transactions performed by all couriers, medical professionals, and/or the like within the particular medical facility would be compared to the same one or more thresholds. The one or more thresholds may also vary between different medical facilities based on the one or more parameters associated with the different medical facilities.

Additionally or alternatively, the analytics engine 110 may receive as an input one or more parameters associated with individuals, such as the couriers, medical professionals, or the like, rather than the medical facility. The one or more parameters associated with the individuals may include a height (e.g., an average height) of the individuals, a stride (e.g., an average stride) of the individuals, or the like. Based on the input of the one or more parameters associated with the individuals, the analytics engine 110 may output one or more thresholds used for one or more medical facilities. Thus, in some instances, trips taken and/or transactions performed by each particular individual within a medical facility would be compared to the different one or more thresholds for each individual. In other instances, the trips taken and/or transactions performed by each particular individual within the medical facility would be compared to the same one or more thresholds.

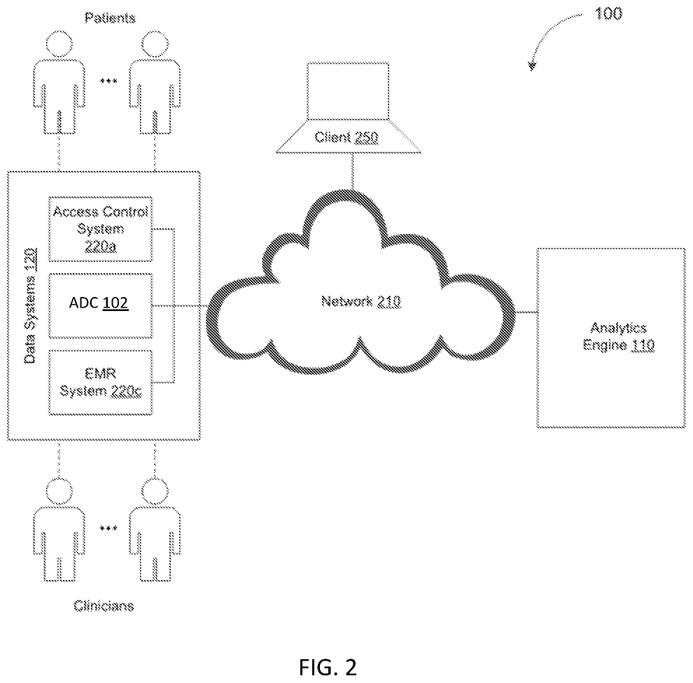

FIG. 2 depicts a system diagram illustrating an example of the trip management system 100, consistent with implementations of the current subject matter. As shown in FIG. 2, the trip management system 100 may include one or more data systems 120, a client 250, and the analytics engine 110.

The analytics engine 110, the client 250, and the one or more data systems 120 may be communicatively coupled via a network 210. The client 250 may be a processor-based device such as, for example, a smartphone, a tablet computer, a wearable apparatus, a desktop computer, a laptop computer, a workstation, and/or the like. The client 250, which may display the generated metrics and/or visualizations, may form a part of or be separately coupled to the one or more data systems, such as the ADC 102 The network 210 may be any wired and/or wireless network including, for example, a public land mobile network (PLMN), a local area network (LAN), a virtual local area network (VLAN), a wide area network (WAN), the Internet, and/or the like.

Referring to FIG. 2, the one or more data systems 120 may include an access control system 220a, an ADC 102, and an electronic medical record (EMR) system 220c. In some implementations, a clinician interacting with the access control system 220a, the ADC 102, and/or the electronic medical record system 220c may trigger the generation of one or more corresponding transaction records, such as during a medical workflow. For example, the clinician dispensing a medication from an ADC may trigger the generation of a transaction record that includes a timestamp, a clinician identifier of the clinician, a device identifier of the dispensing cabinet, a patient identifier of a patient prescribed the medication, an identifier of the medication retrieved from the dispensing cabinet, a quantity of the medication retrieved from the medication cabinet, and/or the like. Additionally or alternatively, as described herein, a medical professional, such as a pharmacy courier interacting with an ADC and/or otherwise performing a transaction at the ADC, may generate one or more transaction records.

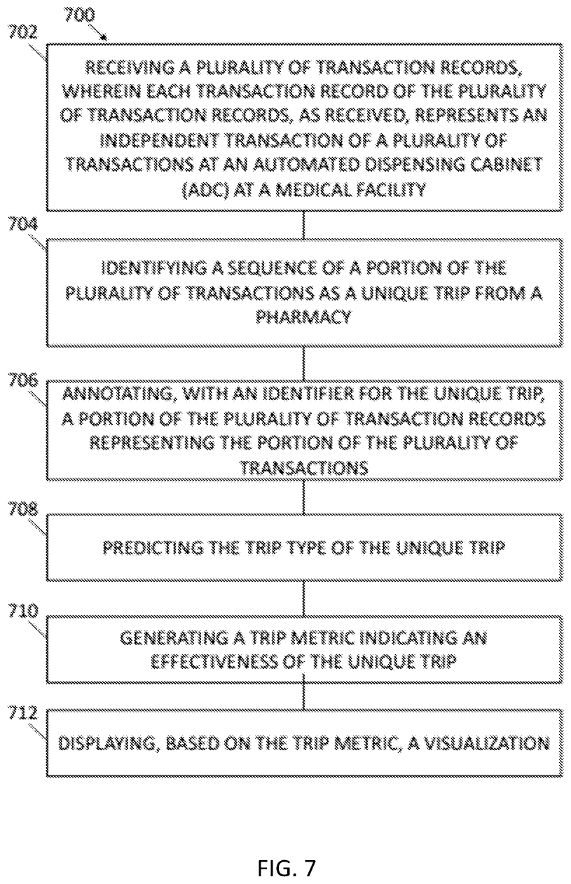

FIG. 7 depicts a flowchart illustrating a process 700 for managing a medical workflow, in accordance with some example implementations. Referring to FIG. 7, the process 700 may be performed at least in part by the analytics engine 110.

At 702, the analytics engine 110 may receive workflow plurality of transaction records. Each transaction record of the plurality of transaction records, as received, may represent an independent transaction of a plurality of transactions at an automated dispensing cabinet, such as the ADC 102, at a medical facility. For example, when a maintenance function, as described herein, is performed at the ADC, the ADC may record one or more corresponding transaction records in a database for each maintenance function. The transaction records may include, among other things, a courier name and identifier, a medication involved, a maintenance function performed (a transaction type), the date/time the transaction occurred, inventory count information, and the like.

For example, as described herein, the one or more transaction records may include time information, such as a start time of a transaction, an end time of a transaction, a length of a transaction, a time the courier left the pharmacy, a time the courier returned to the pharmacy, a length of time between the time the courier left the pharmacy and returned to the pharmacy, and/or the like. The time information may also include the corresponding date. Additionally or alternatively, the one or more transaction records may include one or more transaction values corresponding to a timestamp, a patient identifier, a device identifier, a medical professional identifier, a medication identifier, a prescription order identifier, an inventory information, a patient status, a shift identifier, a location tracking identifier, an infusion information, a compounding information, an administration information, a working off clock indicator, an electronic health record identifier, and/or the like. Additionally or alternatively, the one or more transaction records may include one or more transaction values corresponding to a start time of at least one transaction, a stop or completion time of at least one transaction, a quantity of transactions performed at each ADC during a trip, a quantity of ADCs visited during the medical workflow, an identity of each ADC visited during the medical workflow, an identity of each type of transaction performed at each ADC during a trip, one or more refill parameters, one or more medication removal parameters, and/or the like. In some implementations, the one or more refill parameters may include a number of refills, a number of refills to pockets of the ADC that are empty, a number of refills to pockets of the ADC that are below a minimum PAR level, a number of refills that do not completely refill a corresponding pocket of the ADC to a maximum PAR level, a beginning count of the medication in the pockets of the ADC, an end count of the medication in the pockets of the ADC, a quantity of the medication in the pockets of the ADC, and/or the like. In some implementations, the one or more medication removal parameters includes a quantity of expiry removal attempts from the ADC, a quantity of expiry removal attempts for which no expired items were removed from the ADC, a quantity of expiry removal attempts for which a corresponding pocket of the ADC was emptied, a beginning count of the medication in pockets of the ADC, an end count of the medication in the pockets of the ADC, a quantity of the medication in the pockets of the ADC, and/or the like.

In some implementations, the location tracking identifier may include a patient room number where the transaction occurred, an IP address of the ADC where the transaction occurred, a network IP address (e.g., identifying where the courier's device attached to record the transaction), an identifier for a network access point receiving a message including the transaction, relative or absolute coordinates of a device associated with the transaction (e.g., an ADC or courier device may include coordinates or other location identifier as part of the transaction), or the like.

At 704, the analytics engine 110 may identify a sequence of a portion of the plurality of transactions as a unique trip. The unique trip may be from a pharmacy to one or more ADCs, and/or back to the pharmacy from the one or more ADCs. The identification may be based at least in part on one or more of (i) a comparison of time information (e.g., the start time, the end time, a total time, or the like of a transaction) of or between respective transactions and a threshold inter-transaction trip time and (ii) a comparison of an elapsed time for a portion of the transactions and a threshold elapsed time, a sequence of the portion of the group of transactions as a unique trip.

In some implementations, when the time information is less than or equal to the threshold time, such as the threshold inter-transaction trip time, the analytics engine 110 determines that the transactions are part of the sequence of the portion of the group of transactions defining the unique trip. In some implementations, when the time information is greater than the threshold time, such as the threshold inter-transaction trip time, the analytics engine 110 determines that the transactions are not part of the sequence of the portion of the group of transactions defining the unique trip. Additionally or alternatively, when the elapsed time is less than or equal to the threshold time, such as the threshold elapsed time, the analytics engine 110 determines that the transactions are part of the sequence of the portion of the group of transactions defining the unique trip. Additionally or alternatively, when the elapsed time is greater than the threshold time, such as the threshold elapsed time, the analytics engine 110 determines that the transactions are not part of the sequence of the portion of the group of transactions defining the unique trip.

The time information and the threshold inter-transaction trip time may be a time between sequential transactions (or recorded transaction records). The threshold inter-transaction trip time may be a maximum period of time that would elapse between any two sequential transactions performed by the same courier. The threshold inter-transaction time may be 10 to 30 minutes, 30 to 60 minutes, 60 to 90 minutes, 90 to 120 minutes, longer, or other ranges therebetween. In some implementations, the threshold elapsed time is a total amount of time that elapsed since the start time of a particular transaction. In other words the threshold elapsed time may include a length of the minimum period of time that it would take for a courier to perform a trip (e.g., leave the pharmacy, perform a transaction at one ADC, and return to the pharmacy). The threshold elapsed time may be 10 to 30 minutes, 30 to 60 minutes, 60 to 90 minutes, 90 to 120 minutes, longer, or other ranges therebetween. In some implementations, the identification is based at least in part on a comparison of time information of or between respective transactions and a threshold inter-station time. The threshold inter-station time may be an amount of time elapsed between sequential transactions at different ADCs. The threshold inter-station time may be 10 to 30 minutes, 30 to 60 minutes, 60 to 90 minutes, 90 to 120 minutes, longer, or other ranges therebetween.

Additionally or alternatively, the analytics engine 110 may identify a sequence of a portion of the plurality of transactions as a unique trip when the system receives a recorded time the courier left the pharmacy and a recorded time the courier returned to the pharmacy. In such implementations, the analytics engine 110 may compare the time information (e.g., when the courier left and returned to the pharmacy and/or the length of time between the recorded times) to a threshold amount of time. The threshold inter-station time may be 10 to 30 minutes, 30 to 60 minutes, 60 to 90 minutes, 90 to 120 minutes, longer, or other ranges therebetween. In some implementations, when the time information is less than or equal to the threshold time, such as the threshold amount of time, the analytics engine 110 determines that the transactions are part of the sequence of the portion of the group of transactions defining the unique trip. In some implementations, when the time information is greater than the threshold time, such as the threshold inter-transaction trip time, the analytics engine 110 determines that the transactions are not part of the sequence of the portion of the group of transactions defining the unique trip.

At 706, the analytics engine 110 may annotate, with an identifier for the unique trip, the portion of the plurality of transaction records representing the portion of the plurality of transactions. The identifier may include a numerical, an alphabetical, or an alphanumerical identifier that is associated with the portion of the plurality of transaction records. The identifier may be stored in the database with the transaction records.

At 708, the analytics engine 110 may predict a trip type of the unique trip. For example, based on the identification of the unique trip and/or a model (e.g., a statistical model, a trained machine learning model, etc.) that accepts a set of input values characterizing a trip and provides a set of output values identifying a trip type of the trip, the analytics engine 110 may predict the trip type of the unique trip. The trip type may include a scheduled trip, an unscheduled or ad hoc trip, an undetermined trip, or the like.

In some implementations, the analytics engine 110 may annotate, with an identifier of the trip type, at least one of the portion of the plurality of transaction records and a record for the unique trip. The identifier may include a numerical, an alphabetical, or an alphanumerical identifier that is associated with the portion of the plurality of transaction records. The identifier may be stored in the database with the transaction records.

In some implementations, the analytics engine 110 may schedule, based at least in part on the trip type, at least a portion of a future trip from the pharmacy to the ADC. In some implementations, the analytics engine 110 may schedule, based at least in part on the trip type, elevator calls, may lock or unlock regions of the patient care areas, and/or the like. Thus, accurately predicting the trip type may help to make future trips more efficiently planned.

In some implementations, the analytics engine 110 may filter, based on the identifier of trip type, a plurality of trip records. For example, each of the plurality of trip records may represent an independent trip. The filtering may include removing or hiding, from the plurality of trip records, at least one trip record corresponding to the identifier of the trip type. In other words, the plurality of trip records may be filtered based on whether the identifier indicates a trip record corresponds to a scheduled, unscheduled, or undetermined trip type.

In some implementations, the analytics engine 110 may determine a total number of trips having a particular trip type over a predetermined period of time (e.g., hours, days, weeks, etc.). The analytics engine 110 may detect that the total number of trips over the period of time is greater than or equal to a threshold number of trips. Based upon that determination, the analytics engine 110 may cause generation of an alert indicating that the total number of trips over the period of time is greater than the threshold number of trips. This may indicate that there is a scheduling issue, such as when a large number of ad hoc or unscheduled trips occur. Thus, the analytics engine 110 may adjust, based on the detection, a schedule of a future trip or plurality of future trips.

At 710, the analytics engine 110 may generate, based on the portion of (or entirety of) the plurality of transaction records, at least one trip metric. The trip metric may indicate an effectiveness of the unique trip (or plurality of trips) that are part of a medical workflow. In some implementations, the trip metric indicates the nature of the trip, an urgency of the trip, and/or the like.

In some implementations, the at least one trip metric includes a duration of at least one trip, a quantity of ADCs visited during at least one trip, a quantity of a transaction type of at least one transaction performed at the ADC during a trip, a quantity of different transaction types of the transactions performed at the ADC during at least one trip, a total quantity of trips in the medical workflow, a total quantity of refill transactions performed during the medical workflow, a total quantity of critical low refill transactions performed during the medical workflow, a total quantity of refills less than maximum transactions performed during the medical workflow, a total quantity of inventory transactions performed during the medical workflow, a total quantity of load and/or unload transactions performed during the medical workflow, a total quantity of expiration no-remove transactions performed during the medical workflow, a total quantity of expiration remove transactions performed during the medical workflow, a total duration of the medical workflow, a total quantity of transactions performed during the medical workflow, a total quantity of stock out refills transactions performed during the medical workflow, and/or the like. The at least one trip metric may allow for more efficient planning of the medical workflow and/or a reduction in a number of ad hoc trips that are part of the medical workflow.

In some implementations, the trip metric may be determined based on one or more data models representing the portion of (or entirety of) the plurality of transaction records. The one or more data models may be a parameterized equation that accepts a set of values from a transaction record to generate one or more output values indicating trip association or trip type. The one or more data models may be a machine learning model that accepts a set of values from one or more transaction records via an input layer and provides a set of output values indicating a likelihood that the records are part of a common trip. In some implementations, the one or more data models may accept a trip and a candidate transaction. The one or more data models may then provide a set of output values indicating a likelihood that the candidate transaction is part of the identified trip. One or more data models accepting trip information as an input may provide an additional or alternative set of output values indicating a type for the trip. The output values may be represented as a vector of values where each element of the vector element correspond to a trip type and the value for the vector element corresponds to a likelihood that the trip is of the associated type.

In some implementations, the generation of the at least one trip metric includes applying one or more statistical analysis techniques and/or trained machine learning models to the received data. For example, the analytics engine 110 may apply a machine learning model trained to determine whether at least one trip of the plurality of trips is a scheduled trip or an ad hoc trip. In some implementations, the analytics engine 110 applies a machine-learning model trained to identify an approximate time at which the scheduled trip occurred, and identify the approximate time at which the scheduled trip occurred. In some implementations, the analytics engine may determine, based on the identification of the approximate time at which the scheduled trip occurred, the ad-hoc trip. This allows for a reduction in the quantity of ad hoc trips that form a part of the medical workflow and results in an improved and more efficient medical workflow.

The at least one trip metric may be a comparative metric for the trip or series of trips. For example, the at least one trip metric may identify a trend between two observation periods. The trend identified may include a change in the number of ad-hoc trips. The trend may include a change in the total number of trips. The trend may include a change in the number and type of transactions performed during scheduled trips. In each case, the magnitude of the change may be compared to a threshold to determine the effectiveness of any system or scheduling changes between observation periods.

At 712, the analytics engine 110 may display and/or generate, based on the at least one trip metric, the predicted trip type, the identification of the unique trip, and/or the one or more transaction records, a visualization (such as via the client 250). The visualization may indicate an effectiveness of the medical workflow including the plurality of trips, such as the unique trip. The visualization may additionally or alternatively be used to illustrate a frequency at which various transactions are performed. The visualization may additionally or alternatively be used, by for example, the analytics engine 110 and/or the medical professional, to determine whether a trip is a scheduled trip or an ad hoc trip. The visualization may additionally or alternatively suggest and inform couriers when generating an optimal scheduling of the plurality of trips. The visualization may additionally or alternatively assist couriers and/or the analytics engine 110 in determining whether an ad hoc trip is necessary. The visualization may additionally or alternatively assist couriers and/or the analytics engine 110 in determining whether expired medications are likely to be contained within the pocket of the ADC and which pockets have highest need for an ad hoc or scheduled trip. The visualization may be used to identify trips, trip types, or scheduling recommendations by identifying specific points on the visualization such a slope, inflection point, or other pattern. In some implementations, the data used to generate the visualization may be used in addition to or as an alternative to the graphic.

In some implementations, the analytics engine 110 may additionally or alternatively apply one or more statistical analysis techniques and/or machine learning models to the received at least one transaction record, use the generated at least one trip metric, and/or use the generated and/or displayed visualization, to trigger an investigative workflow, suggest an updated medical workflow, schedule at least a portion of the medical workflow, distinguish between a scheduled trip and an ad hoc trip, distinguish between different types of couriers participating in the medical workflow, detect a diversion of medication during a medical workflow, and/or the like. Thus, the analytics engine 110 may allow for updated, improved, and more efficient medical workflows including a plurality of trips.

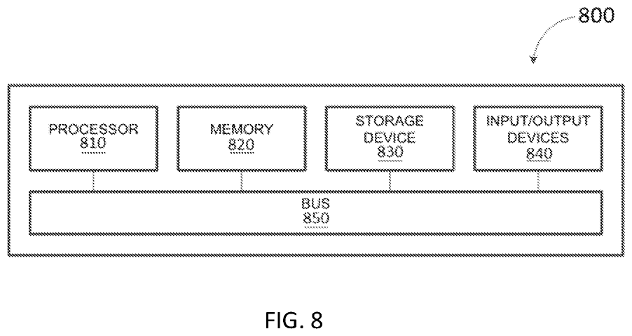

FIG. 8 depicts a block diagram illustrating a computing system 800 consistent with implementations of the current subject matter. Referring to FIGS. 1-7, the computing system 800 can be used to implement the trip management system 100, such as the analytics engine 110, and/or any components therein.

As shown in FIG. 8, the computing system 800 can include a processor 810, a memory 820, a storage device 830, and input/output device 840. The processor 810, the memory 820, the storage device 830, and the input/output device 840 can be interconnected via a system bus 850. The processor 810 is capable of processing instructions for execution within the computing system 800. Such executed instructions can implement one or more components of, for example, the analytics engine 110 and/or the trip management system 100. In some example implementations, the processor 810 can be a single-threaded processor. Alternatively, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, an optical disk device, a tape device, a solid-state device, and/or any other suitable persistent storage means. The input/output device 840 provides input/output operations for the computing system 800. In some example implementations, the input/output device 840 includes a keyboard and/or pointing device. In various implementations, the input/output device 840 includes a display unit for displaying graphical user interfaces.

According to some example implementations, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example implementations, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1-70. (canceled)

71. A system comprising:
at least one data processor; and
at least one memory storing instructions which, when executed by the at least one data processor, result in operations comprising:
receiving a plurality of transaction records, wherein each transaction record of the plurality of transaction records, as received, represents an independent transaction of a plurality of transactions at an automated dispensing cabinet (ADC) at a medical facility; wherein the plurality of transaction records comprises: time information between respective transactions of the plurality of transaction; and an elapsed time for a portion of the plurality of transactions;
identifying, based at least in part on: (i) a comparison of the time information between respective transactions of the plurality of transactions and a threshold inter-transaction trip time; and (ii) a comparison of the elapsed time for the portion of the plurality of transactions and a threshold elapsed trip time, a sequence of the portion of the plurality of transactions as a unique trip from a pharmacy; and
generating, based on the portion of the plurality of transaction records, at least one trip metric indicating an effectiveness of the unique trip; and
suggesting, based on the at least one trip metric, an updated medical workflow configured to reduce a quantity of unscheduled trips of the plurality of trips of the medical workflow, the updated medical workflow comprising one or more of: a sequence of the plurality of trips of the medical workflow, a time at which the plurality of trips should be performed, one or more transactions to perform during each of the plurality of trips, or a route through the medical facility.

72. The system of claim 71, wherein the operations further comprise: predicting, based on the identification of the unique trip and a model that accepts a set of input values characterizing a trip and provides a set of output values identifying a trip type of the trip, the trip type of the unique trip, wherein the trip type of the unique trip is at least one of a scheduled trip or an unscheduled trip.

73. The system of claim 72, wherein the operations further comprise: annotating, with an identifier of the trip type, at least one of the portion of the plurality of transaction records and a record for the unique trip.

74. The system of claim 72, wherein the operations further comprise:
determining a total number of trips having the trip type over a predetermined period of time;
detecting that the total number of trips over the predetermined period of time is greater than or equal to a threshold number of trips; and
generating an alert indicating that the total number of trips over the predetermined period of time is greater than the threshold number of trips.

75. The system of claim 74, wherein the operations further comprise: adjusting, based on the detection, a schedule of a future trip.

76. The system of claim 71, wherein the operations further comprise:
causing presentation of a first plot of trips representing a plurality of trips at the medical facility, the plurality of trips comprising the unique trip;
receiving a message requesting a second plot of trips showing at least two trip types for the medical facility, the at least two trip types comprising an unscheduled trip and a scheduled trip;
predicting, based on the identification of the unique trip and a model that accepts a set of input values characterizing a trip and provides a set of output values identifying a trip type for of the trip, the trip type of each trip of the plurality of trips; and
causing presentation of the second plot of trips, the second plot of trips comprising: a first visualization for at least one trip of the plurality of trips identified as the unscheduled trip; and a second visualization for at least one trip of the plurality of trips identified as the scheduled trip.

77. The system of claim 71, wherein the identifying further comprises: dynamically generating, based on one or more of historical trip data, a size of the medical facility, a number of patient rooms at the medical facility, a number of ADCs at the medical facility, a distance from a pharmacy to a farthest possible location of a transaction of the plurality of transactions, a square footage of the medical facility, a number of patients being treated at the medical facility, a number of elevators at the medical facility, and a number of floors at the medical facility, at least one of the inter-transaction threshold or the threshold elapsed trip time.

78. The system of claim 71, wherein the operations further comprise:
displaying, based on the at least one trip metric, a visualization indicating the effectiveness of the medical workflow comprising a plurality of trips, the plurality of trips comprising the unique trip.

79. The system of claim 72, wherein the model comprises a machine-learning model trained to identify the trip type.

80. The system of claim 71, wherein the at least one trip metric comprises one or more of: a duration of at least one trip of the plurality of trips, a quantity of ADCs visited during the at least one trip, a transaction type of the transaction performed at the ADC during the at least one trip, and a quantity of different transaction types of the transaction performed at the ADC during the at least one trip, a total quantity of trips in the medical workflow, a total quantity of refill transactions performed during the medical workflow, a total quantity of critical low refill transactions performed during the medical workflow, a total quantity of refills less than maximum transactions performed during the medical workflow, a total quantity of inventory transactions performed during the medical workflow, a total quantity of load and/or unload transactions performed during the medical workflow, a total quantity of expiration no-remove transactions performed during the medical workflow, a total quantity of expiration remove transactions performed during the medical workflow, a total duration of the medical workflow, a total quantity of transactions performed during the medical workflow, and a total quantity of stock out refills transactions performed during the medical workflow.

81. The system of claim 78, wherein the updated medical workflow comprises a route for a robot to deliver a medication to the ADC.

82. The system of claim 71, wherein the plurality of transaction records are generated from one or more data systems comprising the ADC, an access control system, or an infusion system, and wherein transaction records are generated from one or more of the ADC and an identification tag of a medical professional performing the independent transaction.

83. The system of claim 72, wherein the predicting comprises: determining an inflection value from the set of input values.

84. The system of claim 83, wherein the predicting further comprises: determining that the unique trip is the scheduled trip when the inflection value is greater than or equal to a threshold value; and determining that the unique trip is the ad hoc trip when the inflection value is less than the threshold value.

85. The system of claim 71, further comprising the ADC.

86. A method comprising:
under control of one or more processing devices,
receiving a plurality of transaction records, wherein each transaction record of the plurality of transaction records, as received, represents an independent transaction of a plurality of transactions at an automated dispensing cabinet (ADC) at a medical facility, wherein the plurality of transaction records comprises: time information between respective transactions of the plurality of transaction; and an elapsed time for a portion of the plurality of transactions;
identifying, based at least in part on: (i) a comparison of the time information between respective transactions of the plurality of transactions and a threshold inter-transaction trip time; and (ii) a comparison of the elapsed time for the portion of the plurality of transactions and a threshold elapsed trip time, a sequence of the portion of the plurality of transactions as a unique trip from a pharmacy; and
predicting, based on the identification of the unique trip and a model that accepts a set of input values characterizing a trip and provides a set of output values identifying a trip type of the trip, the trip type of the unique trip, wherein the trip type of the unique trip is at least one of a scheduled trip or an unscheduled trip; and
generating, based on the portion of the plurality of transaction records, at least one trip metric indicating an effectiveness of the unique trip, wherein the at least one trip metric comprises one or more of: a duration of at least one trip of the plurality of trips, a quantity of ADCs visited during the at least one trip, a transaction type of the transaction performed at the ADC during the at least one trip, and a quantity of different transaction types of the transaction performed at the ADC during the at least one trip, a total quantity of trips in the medical workflow, a total quantity of refill transactions performed during the medical workflow, a total quantity of critical low refill transactions performed during the medical workflow, a total quantity of refills less than maximum transactions performed during the medical workflow, a total quantity of inventory transactions performed during the medical workflow, a total quantity of load and/or unload transactions performed during the medical workflow, a total quantity of expiration no-remove transactions performed during the medical workflow, a total quantity of expiration remove transactions performed during the medical workflow, a total duration of the medical workflow, a total quantity of transactions performed during the medical workflow, and a total quantity of stock out refills transactions performed during the medical workflow.

87. The method of claim 86, further comprising: annotating, with an identifier of the trip type, at least one of the portion of the plurality of transaction records and a record for the unique trip.

88. The method of claim 86, further comprising: scheduling, based in part on the trip type, at least a portion of a future trip from the pharmacy to the ADC.

89. The method of claim 86, further comprising: filtering, based on the identifier of the trip type, a plurality of trip records, wherein each of the plurality of trip records represents an independent trip.

90. The method of claim 86, further comprising:
determining a total number of trips having the trip type over a predetermined period of time;
detecting that the total number of trips over the predetermined period of time is greater than or equal to a threshold number of trips; and
generating an alert indicating that the total number of trips over the predetermined period of time is greater than the threshold number of trips.

91. The method of claim 86, further comprising:
causing presentation of a first plot of trips representing a plurality of trips at the medical facility, the plurality of trips comprising the unique trip;
receiving a message requesting a second plot of trips showing at least two trip types for the medical facility, the at least two trip types comprising an unscheduled trip and a scheduled trip; and
causing presentation of the second plot of trips, the second plot of trips comprising: a first visualization for at least one trip of the plurality of trips identified as the unscheduled trip; and a second visualization for at least one trip of the plurality of trips identified as the scheduled trip.

92. The method claim 86, further comprising:
displaying, based on the at least one trip metric, a visualization indicating the effectiveness of a medical workflow comprising a plurality of trips, the plurality of trips comprising the unique trip.

93. The method of claim 86, further comprising: suggesting, based on the at least one trip metric, an updated medical workflow configured to reduce a quantity of unscheduled trips of the plurality of trips of the medical workflow, wherein the updated medical workflow comprising one or more of: a sequence of the plurality of trips of the medical workflow, a time at which the plurality of trips should be performed, one or more transactions to perform during each of the plurality of trips, and a route through the medical facility.

94. The method of claim 86, wherein the updated medical workflow comprises a route for a robot to deliver a medication to the ADC.

95. The method of claim 87, wherein the predicting comprises: determining an inflection value from the set of input values; determining that the unique trip is the scheduled trip when the inflection value is greater than or equal to a threshold value; and determining that the unique trip is the ad hoc trip when the inflection value is less than the threshold value.
